(19) United States
(12) Patent Application Publication
(10) Pub. No.: US 2024/0417522 A1
(43) Pub. Date: Dec. 19, 2024

(54) METHOD FOR CROSSLINKING HYALURONIC ACID USING RESONANT ACOUSTIC MIXING

(71) Applicant: STUDIO BIOSCIENCE INC., Montréal (CA)

(72) Inventor: Felix POLYAK, Montreal (CA)

(21) Appl. No.: 18/699,854

(22) PCT Filed: Mar. 29, 2023
(86) PCT No.: PCT/CA2023/050420

§ 371 Date: Apr. 9, 2024

Related U.S. Application Data
(60) Provisional application No. 63/362,057, filed on Mar. 29, 2022.

(51) Int. Cl. C08J 3/24; A61K 31/738; A61L 27/20; A61L 27/52; B01F 23/50; B01F 101/22; C08B 37/08; C08J 3/075; C08J 3/28

(52) U.S. Cl. CPC C08J 3/24; A61K 31/738; A61L 27/20; A61L 27/52; B01F 23/511; B01F 23/551; C08B 37/0072; C08J 3/075; C08J 3/28; A61L 2430/34; B01F 2101/22; B01F 2215/0454; B01F 2215/0477; C08J 2305/08

(57) ABSTRACT

A process for crosslinking a polymer using resonant acoustic mixing is disclosed herein. The process comprises adding an aqueous solution comprising a dissolved base to the polymer to produce a substantially homogenous gel; adding a crosslinking agent to the substantially homogeneous gel to produce a mixture; and subjecting the mixture to resonant acoustic mixing conditions sufficient to effect crosslinking of the polymer, wherein the resonant acoustic mixing conditions comprise a forcing energy ranging between about 20 g to about 100 g. Also disclosed are cosmetic, therapeutic and/or prophylactic applications of products comprising a crosslinked polymer produced by resonant acoustic mixing.

### CROSS REFERENCE TO RELATED APPLICATIONS

This application claims the benefit of U.S. Provisional Application 63/362,057, filed Mar. 29, 2022. The contents of the referenced application are incorporated into the present application by reference.

### BACKGROUND

### 1. Field

This disclosure relates to the field of chemistry. More specifically, but not exclusively, the present disclosure broadly relates to the crosslinking of carbohydrate polymers using resonant acoustic mixing (RAM). Yet more specifically, but not exclusively, the present disclosure broadly relates to the crosslinking of hyaluronic acid using resonant acoustic mixing.

### 2. Related Art

Hyaluronic acid (HA) is a natural biopolymer that possesses many important biological functions covering a broad range of applications. The crosslinking of hyaluronic acid is central to making it biologically and chemically more stable. Moreover, crosslinking also imparts improved chemical, physical and rheological properties. Crosslinked hyaluronic acid has been used for various medical and cosmetic applications.

Hyaluronic acid exhibits a half-life of a few days in human tissue. Many hyaluronic acid-based products intended for medical and/or cosmetic use comprise chemically modified hyaluronic acid. The chemical modification typically consists of crosslinking the hyaluronic acid. It has been shown that crosslinked hyaluronic acid exhibits a longer half-life in human tissue. Indeed, many of the commercially available hyaluronic acid-based dermal fillers comprise crosslinked hyaluronic acid. The crosslinking is typically achieved using bifunctional crosslinking reagents such as 1,4-butanediol diglycidyl ether (BDDE). Such dermal fillers are commonly used in many aesthetic medicinal applications such as for facial rejuvenation. Typically, the crosslinking is achieved by first preparing a hyaluronic acid gel by adding the hyaluronic acid to an aqueous sodium hydroxide solution. An aqueous sodium hydroxide solution comprising BDDE is subsequently added, and the reaction mixture incubated over a period of several hours, typically at about 50° C. Some of the more common commercially available BDDE-crosslinked hyaluronic acid products include CPM® (Cohesive Polydensified Matrix), NASHA® (nonanimal stabilized hyaluronic acid), Hylacross®, Vycross®, RHAR (Resilient Hyaluronic Acid), and XpresHAn®.

At present, most of the currently available methods for producing crosslinked hyaluronic acid produce gels having low to moderate viscosities, require reaction times of several hours, and require reaction temperatures of about 50° C. which can lead to mild to moderate degradation of the hyaluronic acid. A novel method for producing crosslinked hyaluronic acid having a broader range of viscosities and having improved rheological properties, while substantially avoiding degradation, is of commercial interest.

### SUMMARY

The present disclosure broadly relates to the crosslinking of carbohydrate polymers using resonant acoustic mixing. More specifically, but not exclusively, the present disclosure broadly relates to the crosslinking of hyaluronic acid using resonant acoustic mixing. Yet more specifically, the present disclosure broadly relates to the crosslinking of hyaluronic acid with a bifunctional crosslinking agent using resonant acoustic mixing. Yet more specifically, the present disclosure broadly relates to the crosslinking of hyaluronic acid with 1,4-butanediol diglycidyl ether (BDDE) using resonant acoustic mixing.

In an aspect, the present disclosure relates to a process for crosslinking a polymer using resonant acoustic mixing, the process comprising: adding the polymer to an aqueous solution comprising a dissolved base to produce a substantially homogenous gel; adding a crosslinking agent to the substantially homogeneous gel to produce a mixture; and subjecting the mixture to resonant acoustic mixing conditions sufficient to effect crosslinking of the polymer; wherein the resonant acoustic mixing conditions comprise a forcing energy ranging between about 20 g to about 100 g. In an embodiment of the present disclosure, the resonant acoustic forcing energy ranges between about 60 g to about 80 g. In an embodiment of the present disclosure, the addition steps are carried out at room temperature. In an embodiment of the present disclosure, the resonant acoustic mixing step is carried out at room temperature. In an embodiment of the present disclosure, at least one of the process steps is carried out at room temperature. In an embodiment of the present disclosure, the process further comprises a purification step and/or sterilization step. In some embodiments, the purification step comprises washing the crosslinked polymer using an aqueous hydrochloric acid solution.

In an aspect of the present disclosure, the resonant acoustic mixing conditions further comprise mixing frequencies ranging between about 40 Hz to about 90 Hz. In an embodiment of the present disclosure, the mixing frequency is about 60 Hz.

In an aspect of the present disclosure, the polymer for crosslinking is a carbohydrate polymer or a salt thereof. In an embodiment of the present disclosure, the carbohydrate polymer is hyaluronic acid or a salt thereof.

In an aspect of the present disclosure, the crosslinking agent is a bifunctional crosslinking agent. In an embodiment of the present disclosure, the bifunctional crosslinking agent comprises 1,4-butanediol diglycidyl ether (BDDE).

In an aspect of the present disclosure, the dissolved base comprises sodium hydroxide.

In an aspect of the present disclosure, the resonant acoustic mixing is carried out over a period of time ranging from about 1 minute to about 10 minutes.

In an aspect, the present disclosure relates to a product comprising a crosslinked polymer produced by resonant acoustic mixing. In an embodiment of the present disclosure, the crosslinked polymer is crosslinked hyaluronic acid. In an embodiment of the present disclosure, the product is a dermal filler. In an embodiment of the present disclosure, the dermal filler is an injectable dermal filler. In an embodiment of the present disclosure, the dermal filler is for use in cosmetic applications. In an embodiment of the present disclosure, the cosmetic applications comprise treating and/or reducing the appearance of fine lines or wrinkles, glabellar lines, nasolabial folds, chin folds, marionette lines, jawlines, perioral wrinkles, crow's feet, oral commissures, cutaneous depressions, scars, temples, malar and buccal fat pads, tear troughs or facial asymmetries. In an embodiment of the present disclosure, the dermal filler is for subdermal support of the brows, nose, lips, cheeks, chin, perioral region and/or infraorbital region. In an embodiment of the present disclosure, the dermal filler is for therapeutic and/or prophylactic applications. In an embodiment of the present disclosure, the therapeutic and/or prophylactic applications comprise stress urinary incontinence, vesicoureteral reflux (VUR), vocal fold insufficiency, and vocal fold medialization.

In an aspect, the present disclosure relates to a method for treating a biological tissue or for increasing the volume of a biological tissue, the method comprising administering to a subject in need thereof an effective amount of an injectable dermal filler comprising a crosslinked polymer produced by resonant acoustic mixing.

In an aspect, the present disclosure relates to a process for crosslinking a polymer using resonant acoustic mixing, the process comprising: adding an aqueous solution comprising a dissolved base to the polymer to produce a substantially homogenous gel; adding a crosslinking agent to the substantially homogeneous gel to produce a mixture; and subjecting the mixture to resonant acoustic mixing conditions sufficient to effect crosslinking of the polymer; wherein the resonant acoustic mixing conditions comprise a forcing energy ranging between about 20 g to about 100 g.

Also disclosed in the context of the present disclosure are embodiments 1 to 46. Embodiment 1 is a process for crosslinking a polymer using resonant acoustic mixing, the process comprising: adding an aqueous solution comprising a dissolved base to the polymer to produce a substantially homogenous gel; adding a crosslinking agent to the substantially homogeneous gel to produce a mixture; and subjecting the mixture to resonant acoustic mixing conditions sufficient to effect crosslinking of the polymer; wherein the resonant acoustic mixing conditions comprise a forcing energy ranging between about 20 g to about 100 g. Embodiment 2 is the process of embodiment 1, wherein the resonant acoustic mixing conditions further comprise mixing frequencies ranging between about 40 Hz to about 90 Hz. Embodiment 3 is the process of embodiment 2, wherein the mixing frequency is about 60 Hz. Embodiment 4 is the process of any one of embodiments 1 to 3, wherein the resonant acoustic forcing energy ranges between about 60 g to about 80 g. Embodiment 5 is the process of any one of embodiments 1 to 4, wherein the substantially homogenous gel is produced following resonant acoustic mixing. Embodiment 6 is the process of embodiment 5, wherein the resonant acoustic mixing comprises a forcing energy ranging between about 20 g to about 100 g and mixing frequencies ranging between about 40 Hz to about 90 Hz. Embodiment 7 is the process of embodiment 6, wherein the mixing frequency is about 60 Hz. Embodiment 8 is the process of any one of embodiments 1 to 7, wherein the resonant acoustic forcing energy ranges between about 60 g to about 80 g. Embodiment 9 is the process of any one of embodiments 1 to 8, wherein the polymer is a carbohydrate polymer or a salt thereof. Embodiment 10 is the process of embodiment 9, wherein the carbohydrate polymer is hyaluronic acid or a salt thereof. Embodiment 11 is the process of any one of embodiments 1 to 10, wherein the crosslinking agent is a bifunctional crosslinking agent. Embodiment 12 is the process of any one of embodiment 11, wherein the bifunctional crosslinking agent comprises 1,4-butanediol diglycidyl ether (BDDE). Embodiment 13 is the process of any one of embodiments 1 to 12, wherein the dissolved base comprises sodium hydroxide. Embodiment 14 is the process of any one of embodiments 1 to 13, wherein the addition steps are carried out at room temperature. Embodiment 15 is the process of embodiment 1, wherein the resonant acoustic mixing step is carried out at room temperature. Embodiment 16 is the process of any one of embodiments 1 to 15, wherein at least one of the process steps is carried out at room temperature. Embodiment 17 is the process of embodiment 1, wherein the resonant acoustic mixing is carried out over a period of time ranging from about 1 minute to about 10 minutes. Embodiment 18 is the process of any one of embodiments 1 to 17, further comprising a purification step and/or sterilization step. Embodiment 19 is the process of embodiment 18, wherein the purification step comprises washing the crosslinked polymer using an aqueous hydrochloric acid solution.

Embodiment 20 is a product comprising a crosslinked polymer produced by resonant acoustic mixing. Embodiment 21 is the product of embodiment 20, wherein the crosslinked polymer is crosslinked hyaluronic acid. Embodiment 22 is the product of embodiment 20 or 21, wherein the product is a dermal filler. Embodiment 23 is the product of embodiment 22, wherein the dermal filler is an injectable dermal filler.

Embodiment 24 is the use of the injectable dermal filler as defined in embodiment 23 for cosmetic applications. Embodiment 25 is the use of embodiment 24, wherein the cosmetic applications comprise treating and/or reducing the appearance of fine lines or wrinkles, glabellar lines, nasolabial folds, chin folds, marionette lines, jawlines, perioral wrinkles, crow's feet, oral commissures, cutaneous depressions, scars, temples, malar and buccal fat pads, tear troughs and facial asymmetries.

Embodiment 26 is the use of the injectable dermal filler as defined in embodiment 23 for subdermal support of the brows, nose, lips, cheeks, chin, perioral region and/or infraorbital region.

Embodiment 27 is the use of the product as defined in embodiment 20 for therapeutic and/or prophylactic applications. Embodiment 28 is the use of embodiment 27, wherein the therapeutic and/or prophylactic applications comprise stress urinary incontinence, vesicoureteral reflux (VUR), vocal fold insufficiency, and vocal fold medialization.

Embodiment 29 is a method for treating a biological tissue or for increasing the volume of the biological tissue, the method comprising administering to a subject in need thereof an effective amount of the injectable dermal filler as defined in embodiment 23.

Embodiment 30 is a process for crosslinking a polymer using resonant acoustic mixing, the process comprising subjection a mixture comprising the polymer and a crosslinking agent to resonant acoustic mixing conditions sufficient to effect crosslinking of the polymer. Embodiment 31 is the process of embodiment 30, further comprising adding an aqueous solution comprising a dissolved base to the polymer followed by resonant acoustic mixing to produce a substantially homogenous gel, and adding a crosslinking agent to the substantially homogeneous gel to produce the mixture. Embodiment 32 is the process of embodiment 31, wherein the dissolved base comprises sodium hydroxide. Embodiment 33 is the process of embodiment 30, wherein the resonant acoustic mixing conditions comprise a forcing energy ranging between about 20 g to about 100 g. Embodiment 34 is the process of embodiment 33, wherein the resonant acoustic forcing energy ranges between about 60 g to about 80 g. Embodiment 35 is the process of embodiment 33 or 34, wherein the resonant acoustic mixing conditions further comprise mixing frequencies ranging between about 40 Hz to about 90 Hz. Embodiment 36 is the process of embodiment 35, wherein the mixing frequency is about 60 Hz. Embodiment 37 is the process of any one of embodiments 30 to 36, wherein the polymer is a carbohydrate polymer or a salt thereof. Embodiment 38 is the process of embodiment 37, wherein the carbohydrate polymer is hyaluronic acid or a salt thereof. Embodiment 39 is the process of any one of embodiments 30 to 38, wherein the crosslinking agent is a bifunctional crosslinking agent. Embodiment 40 is the process of embodiment 39, wherein the bifunctional crosslinking agent comprises 1,4-butanediol diglycidyl ether (BDDE). Embodiment 41 is the process of embodiment 30, wherein the resonant acoustic mixing is carried out at room temperature. Embodiment 42 is the process of embodiment 30, wherein the resonant acoustic mixing is carried out over a period of time ranging from about 1 minute to about 10 minutes. Embodiment 43 is the process of any one of embodiments 30 to 42, further comprising a purification step and/or sterilization step. Embodiment 44 is the process of embodiment 43, wherein the purification step comprises washing the crosslinked polymer using an aqueous hydrochloric acid solution.

Embodiment 45 is a gel comprising a crosslinked polymer produced by resonant acoustic mixing. Embodiment 46 is the gel of embodiment 45, wherein the crosslinked polymer is crosslinked hyaluronic acid.

The word “a” or “an” when used in conjunction with the term “comprising” in the claims and/or the specification may mean “one”, but it is also consistent with the meaning of “one or more”, “at least one”, and “one or more than one” unless the content clearly dictates otherwise. Similarly, the word “another” may mean at least a second or more unless the content clearly dictates otherwise.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

As used in this specification and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The foregoing and other advantages and features of the present disclosure will become more apparent upon reading of the following non-restrictive detailed description of illustrative embodiments thereof, with reference to the accompanying drawings/figures. It should be understood, however, that the detailed description and the illustrative embodiments, while indicating specific embodiments of the disclosure, are given by way of illustration only, since various changes and modifications within the spirit and scope of the disclosure will become apparent to those skilled in the art from this description.

### BRIEF DESCRIPTION OF THE DRAWINGS/FIGURES

The following figure(s)/drawing(s) form part of the present specification and are included to further demonstrate certain aspects of the present specification. The present specification may be better understood by reference to one or more of these figure(s)/drawing(s) in combination with the detailed description. In the appended drawing(s)/figure(s):

FIG. 1—Illustration of a dynamic viscosity plot for BDDE-crosslinked hyaluronic acids (ARC-02, ARC-06, ARC-07 and ARC-08) obtained using resonance acoustic mixing in accordance with an embodiment of the present disclosure. ARC-02: Crosslinked HA made from HA starting material having a molecular weight of 2 MDa; BDDE 30% (amount of BDDE relative to the amount of HA starting material); Concentration of HA in final gel product 2.3% (crosslinked and non-crosslinked HA). ARC-06: Crosslinked HA made from HA starting material having a molecular weight of 1 MDa; BDDE 15% (amount of BDDE relative to the amount of HA starting material); Concentration of HA in final gel product 2.3% (crosslinked and non-crosslinked HA). ARC-07: Crosslinked HA made from HA starting material having a molecular weight of 2 MDa; BDDE 15% (amount of BDDE relative to the amount of HA starting material); Concentration of HA in final gel product 2.3% (crosslinked and non-crosslinked HA). ARC-08: Crosslinked HA made from HA starting material having a molecular weight of 3 MDa; BDDE 15% (amount of BDDE relative to the amount of HA starting material); Concentration of HA in final gel product 2.3% (crosslinked and non-crosslinked HA). RHA®1, RHA®2, RHA®3 and RHA®4 (RHA®—Resilient Hyaluronic Acid; Commercial dermal fillers).

FIG. 2—Illustration of an IR spectrum of a non-crosslinked hyaluronic acid gel (2.3% HA in water). The IR spectrum is characterized by the absorption bands typical for non-crosslinked HA at 1635.8 cm⁻¹ (C═O of the N-acetyl amino group) and 3262.4 cm⁻¹ (OH groups).

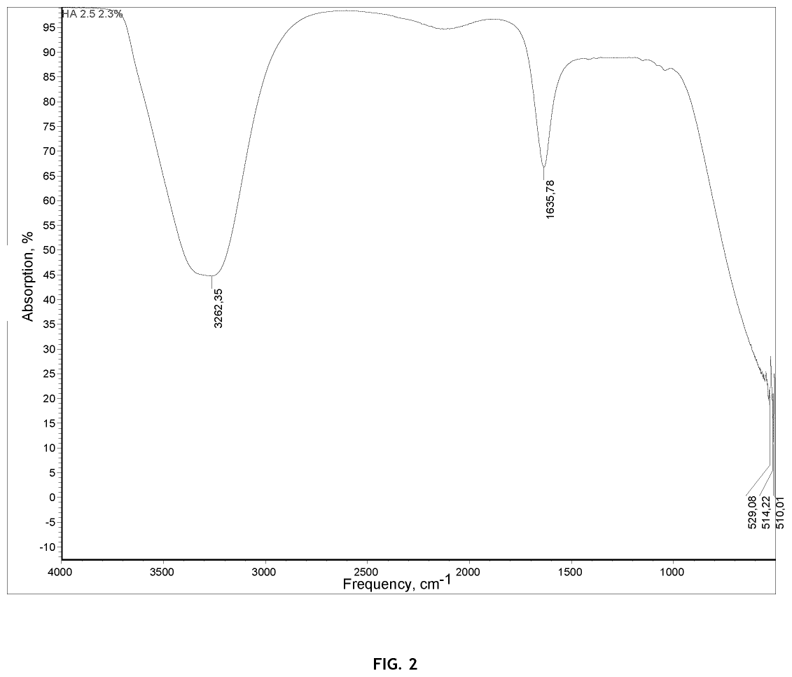

FIG. 3—Illustration of an IR spectrum of a BDDE crosslinked hyaluronic acid obtained by standard chemical crosslinking. The IR spectrum is characterized by the absorption bands typical for crosslinked HA at 1077.14 cm⁻¹ (C—O—C ether; indicative of crosslinking), 1635.56 cm⁻¹ (C═O of the N-acetyl amino group), and 3269.57 cm⁻¹ (OH groups).

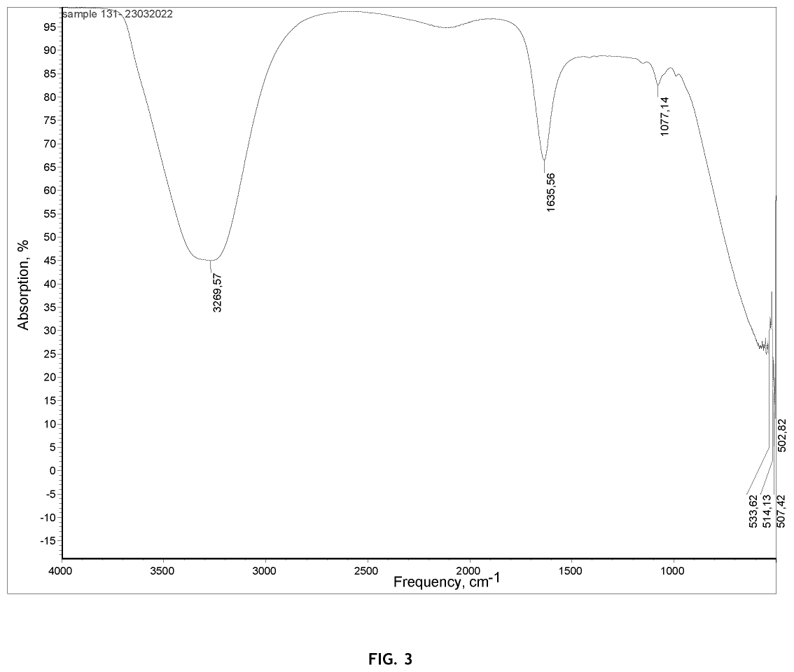

FIG. 4—Illustration of an IR spectrum of an exemplary BDDE-crosslinked hyaluronic acid obtained using resonant acoustic mixing in accordance with an embodiment of the present disclosure. The IR spectrum is characterized by the absorption bands typical for crosslinked HA at 1078.42 cm⁻¹ (C—O—C ether; indicative of crosslinking), 1635.33 cm⁻¹ (C═O of the N-acetyl amino group) and 3265.89 cm⁻¹ (OH groups).

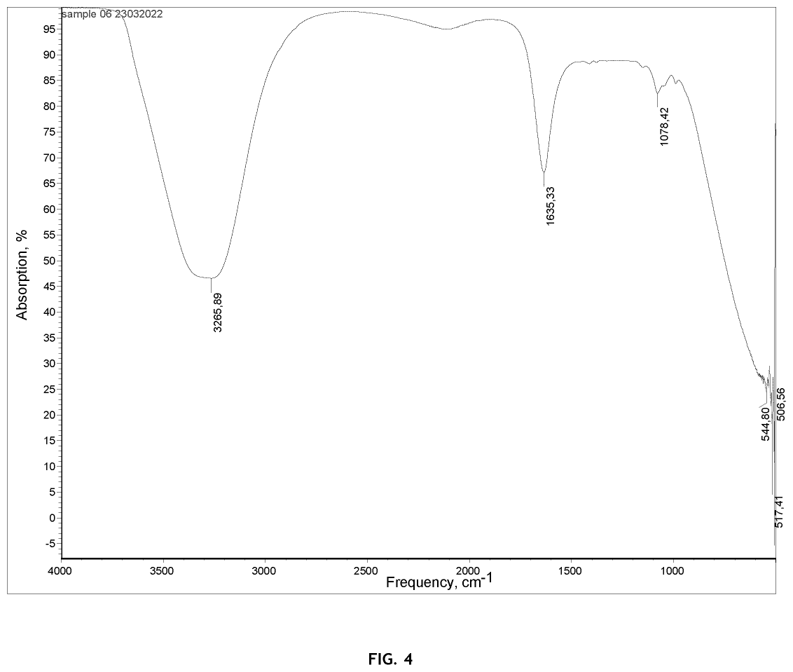

FIG. 5—Illustration of the overlayed IR spectra of FIGS. 2-4. As can be clearly observed, the spectra of BDDE crosslinked hyaluronic acid, obtained by standard chemical crosslinking, and that of BDDE crosslinked hyaluronic acid obtained using resonant acoustic mixing, both exhibit the characteristic C—O—C absorption signal in the 1250-1050 cm⁻¹ region typical of crosslinking.

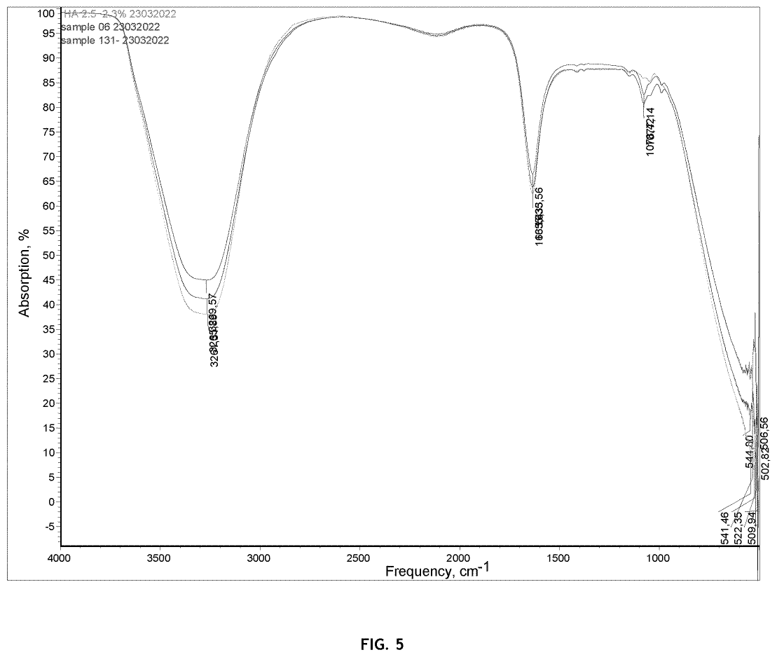

### DETAILED DESCRIPTION OF ILLUSTRATIVE EMBODIMENTS

The present disclosure broadly relates to the crosslinking of carbohydrate polymers using resonant acoustic mixing. More specifically, but not exclusively, the present disclosure broadly relates to the crosslinking of hyaluronic acid using resonant acoustic mixing. Yet more specifically, the present disclosure broadly relates to the crosslinking of hyaluronic acid with a bifunctional crosslinking agent using resonant acoustic mixing. Yet more specifically, the present disclosure broadly relates to the crosslinking of hyaluronic acid with 1,4-butanediol diglycidyl ether (BDDE) using resonant acoustic mixing.

In an aspect, the present disclosure relates to a process for crosslinking a polymer using resonant acoustic mixing, the process comprising: adding the polymer to an aqueous solution comprising a dissolved base to produce a substantially homogenous gel; adding a crosslinking agent to the substantially homogeneous gel to produce a mixture; and subjecting the mixture to resonant acoustic mixing conditions sufficient to effect crosslinking of the polymer; wherein the resonant acoustic mixing conditions comprise a forcing energy ranging between about 20 g to about 100 g. These and other aspects of the disclosure are described in greater detail below.

In an aspect of the present disclosure, a mixture comprising the polymer and the aqueous solution comprising the dissolved base (e.g., sodium hydroxide) is subjected to resonant acoustic mixing to provide a substantially homogeneous gel. In an embodiment, the resonant acoustic mixing is performed at a forcing energy ranging between about 20 g to about 100 g; in a further embodiment between about 25 g to about 95 g; in a further embodiment between about 30 g to about 90 g; in a further embodiment between about 35 g to about 85 g; in a further embodiment between about 40 g to about 80 g; in a further embodiment between about 45 g to about 75 g; in a further embodiment between about 50 g to about 70 g; in a further embodiment between about 55 g to about 65 g; in a further embodiment at about 60 g. In an embodiment, in addition to the forcing energy, the resonant acoustic mixing is performed at a mixing frequency ranging between about 40 Hz to about 90 Hz; in a further embodiment between about 45 Hz to about 85 Hz; in a further embodiment between about 50 Hz to about 80 Hz; in a further embodiment between about 55 Hz to about 75 Hz; in a further embodiment between about 60 Hz to about 70 Hz; in a further embodiment at about 60 Hz. In an embodiment, the resonant acoustic mixing is performed over a period of time ranging from about 1 minute to about 10 minutes; in a further embodiment from about 2 minutes to about 9 minutes; in a further embodiment from about 3 minutes to about 8 minutes; in a further embodiment from about 4 minutes to about 7 minutes; in a further embodiment from about 5 minutes to about 6 minutes; about 1 minute; about 2 minutes; about 3 minutes; about 4 minutes; about 5 minutes; about 6 minutes; about 7 minutes; about 8 minutes; about 9 minutes; or about 10 minutes.

In an aspect of the present disclosure, the substantially homogeneous gel obtained following the resonant acoustic mixing of the polymer and the aqueous solution comprising the dissolved base (e.g., sodium hydroxide), is combined with an aqueous solution of dissolved base (e.g., sodium hydroxide) and a crosslinking agent (e.g., BDDE), and subjected to further resonant acoustic mixing to effect crosslinking of the polymer. In an embodiment, the resonant acoustic mixing is performed at a forcing energy ranging between about 20 g to about 100 g; in a further embodiment between about 25 g to about 95 g; in a further embodiment between about 30 g to about 90 g; in a further embodiment between about 35 g to about 85 g; in a further embodiment between about 40 g to about 80 g; in a further embodiment between about 45 g to about 75 g; in a further embodiment between about 50 g to about 70 g; in a further embodiment between about 55 g to about 65 g; in a further embodiment at about 60 g. In an embodiment, in addition to the forcing energy, the resonant acoustic mixing is performed at a mixing frequency ranging between about 40 Hz to about 90 Hz; in a further embodiment between about 45 Hz to about 85 Hz; in a further embodiment between about 50 Hz to about 80 Hz; in a further embodiment between about 55 Hz to about 75 Hz; in a further embodiment between about 60 Hz to about 70 Hz; in a further embodiment at about 60 Hz. In an embodiment, the resonant acoustic mixing is performed over a period of time ranging from about 1 minute to about 10 minutes; in a further embodiment from about 2 minutes to about 9 minutes; in a further embodiment from about 3 minutes to about 8 minutes; in a further embodiment from about 4 minutes to about 7 minutes; in a further embodiment from about 5 minutes to about 6 minutes; about 1 minute; about 2 minutes; about 3 minutes; about 4 minutes; about 5 minutes; about 6 minutes; about 7 minutes; about 8 minutes; about 9 minutes; or about 10 minutes.

The process for crosslinking a polymer (e.g., a carbohydrate polymer such as hyaluronic acid) using resonant acoustic mixing, advantageously provides for improved process parameters such as reaction time and reaction temperatures. Indeed, the use of resonant acoustic mixing advantageously provides for the crosslinking of a polymer (e.g., a carbohydrate polymer such as hyaluronic acid) in several minutes at room temperature. Performing the crosslinking at room temperature advantageously provides for preventing potential decomposition of the polymer at more elevated temperatures such as 50° C., especially when the crosslinking is performed over extended periods of time such as one or more hours. Decomposition may be especially problematic with carbohydrate polymers such as hyaluronic acid.

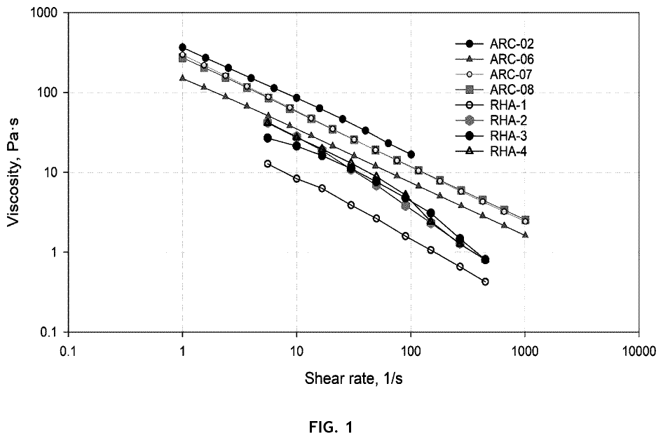

Moreover, in addition to providing for greater reproducibility, the process for crosslinking a polymer (e.g., a carbohydrate polymer such as hyaluronic acid) using resonant acoustic mixing, advantageously provides for crosslinked materials having improved physical and rheological properties. In an embodiment of the present disclosure, the polymer is a carbohydrate polymer, non-limiting examples of which include hyaluronic acid. In an embodiment of the present disclosure, the BDDE-crosslinked hyaluronic acids obtained using resonant acoustic mixing have high dynamic viscosities and a relatively high storage modulus G′ and loss modulus G″ relative to the commercial dermal filers RHA®1, RHA®2, RHA®3 and RHA®4 (FIG. 1). Consequently, the use of resonant acoustic mixing for crosslinking a carbohydrate polymer (e.g., hyaluronic acid) advantageously provides for dermal fillers having improved physical and rheological properties.

As used herein, the term “crosslinking agent” refers to any material that is capable of crosslinking a polymer in accordance with the present disclosure. In an embodiment, the crosslinking agent refers to any material that is capable of crosslinking a hydroxyl polymer (e.g., hyaluronic acid). In a further embodiment of the present disclosure, the crosslinking agent is capable of covalently crosslinking the hydroxyl polymer.

As used herein, the term “viscoelasticity” refers to the property of materials that exhibit both viscous and elastic characteristics when undergoing deformation. More specifically, the property of crosslinked carbohydrate polymers, such as crosslinked hyaluronic acid, suitable for use as dermal fillers that undergo shear deformation. Elasticity is defined as the degree of recovery when a shear force is applied and is subsequently removed. A stronger shear force is typically required in order to deform a material exhibiting high elasticity. A highly viscous material, however, is readily deformed and exhibits low degrees of restoration following the removal of the shear forces.

Five rheological parameters are typically used to describe the viscoelastic properties of a gel-like substance: dynamic viscosity η (Pa/sec) at different shear rates; complex modulus G* (Pa), which provides an indication of the overall viscoelastic properties; storage modulus G′ (Pa), which provides an indication of the elastic properties; loss modulus G″ (Pa), which provides an indication of the viscous properties; and tan δ which is a measure of the ratio of viscous over elastic properties (G″/G′) of a viscoelastic material.

G*, the “complex modulus,” is the total energy required to deform a material using shear stress. This term is commonly referred to as filler “hardness,” and represents the degree of difficulty to alter the shape of an individual cross-linked unit of filler. G* reflects the “hardness” of multiple units of cross-linked HA, not the hardness of the whole gel deposit. It is determined by the following formula: G*=\sqrt{(G′)²+(G″)²}.

G′, the “storage/elastic modulus,” represents the energy fraction of G* stored by the gel during deformation and used to recover its original shape. G′ measures the elastic behavior of a gel or to what degree the gel can recover its original shape after shear deformation forces are removed.

G″, the “loss/viscous modulus,” represents the energy fraction of G* lost due to shear deformation through internal friction. G″ is not directly related to viscosity because the HA filler is not purely viscous. Instead, this term reflects the inability of the gel to completely recover its shape after the shear stress is removed. Clinically, G″ is related to injectability.

Tan δ refers to the elasticity and viscosity of a material. It is a measure of the ratio of viscous to elastic components of G*, defined as tan δ=G″/G′. Tan δ determines whether the material is mainly elastic (tan δ<1), exhibiting a gel-like behavior (e.g., a block of gelatin), or whether it is mainly viscous (tan δ>1), behaving more like a viscous liquid. In cross-linked HA fillers, tan δ is usually low (ranging from 0.05 to 0.80), meaning that the elastic (i.e., gel-like) behavior under low shear stress is dominant over the viscous (i.e., liquid) behavior. Lower tan δ values are usually associated with higher G′ values because HA fillers have low G″ values.

The gel viscosity is a measure of the resistance of a fluid to deformation under shear stress, like the stresses occurring during extrusion or the stresses following application within the tissue of a patient. The higher the viscosity, the greater the gel's resistance to flow. A dermal filler should exhibit high viscosity at low shear forces and low viscosity at high shear forces (“shear thinning behavior”). The shear thinning behavior provides for the gels to easily flow during extrusion (typically associated with high shear forces) while remaining in place after injection (typically associated with low shear forces), avoiding dispersion in the surrounding tissues. Moreover, viscosity values can predict tissue integration patterns. Less viscous gels are expected to spread more into the surrounding tissues following injection (high tissue integration), resulting in a natural-looking, none “palpable” effect, suitable for the treatment of more superficial areas. High viscous gels, however, are better suited for injections at deeper levels.

Dermal fillers should be sufficiently viscoelastic so that they can be readily injected under high strain while also being sufficiently elastic to resist shear deformation forces once injected into soft tissue. Indeed, a purely elastic filler would be almost impossible to inject through a needle as it would require a tremendous amount of force on the plunger to eject it in a nonreversible manner. Similarly, a purely viscous filler would readily and irreversibly deform under even moderate shear forces, and would therefore not retain its shape for any significant amounts of time following injection, even upon removal of the shear forces.

### EXAMPLES

The following examples are included to demonstrate preferred embodiments of the disclosure. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Crosslinking Procedure

Hyaluronic acid (1 g) was added to a flask followed by the addition of an aqueous solution of sodium hydroxide (5 mL; 0.25 N). The flask was subsequently positioned into a RAM (Resonance Acoustic Mixing) instrument set to operate at 60 Hz and 60 G (G-forces or acceleration) and mixed over a period of 3 minutes at room temperature, resulting in the formation of a homogeneous gel. An aqueous solution of sodium hydroxide (1.5 mL; 0.25 N) comprising BDDE (0.14 mL) was then added to the flask. The flask was repositioned into the RAM instrument set to operate 60 Hz and 80 G and mixed over a period of 6 minutes at room temperature. The resulting gel was washed several times using an aqueous hydrochloric acid solution (0.05N), and the pH of the gel adjusted to a range from about 6.8-to about 7.4 using an aqueous solution of sodium monophosphate dibasic. Water was subsequently added to adjust the concentration of the crosslinked hyaluronic acid in the gel to a value ranging from about 1.5% to about 2.6%, and the gel homogenized. Finally, the homogenized gel was sterilized at 121° C. over a period of 15 minutes.

### Example 2—Rheological Measurements

The dynamic viscosity “η”, storage modulus G′, and loss modulus G″ for selected BDDE-crosslinked hyaluronic acids obtained in accordance with an embodiment of the present disclosure using resonant acoustic mixing (i.e., ARC-02, ARC-06, ARC-07, ARC-08) were measured (Tables 1 and 4).

TABLE 1
Storage modulus G′ and loss modulus G″ for
selected BDDE-crosslinked hyaluronic acids.

| | Storage Modulus G′, Pa | | | Loss Modulus G″, Pa | | |
|---|---|---|---|---|---|---|
| | Frequency, Hz | | | Frequency, Hz | | |
| ID | 0.5 | 5 | 10 | 0.5 | 5 | 10 |
| ARC-02 | 1540 | 1810 | 1850 | 217 | 163 | 135 |
| ARC-06 | 320 | 392 | 405 | 65 | 46 | 42 |
| ARC-07 | 520 | 598 | 620 | 66 | 66 | 78 |
| ARC-08 | 454 | 519 | 544 | 56 | 45 | 48 |

### Example 3—Rheological Properties of Crosslinked Hyaluronic Acid Obtained by RAM

The rheological properties of BDDE-crosslinked hyaluronic acid obtained in accordance with an embodiment of the present disclosure using resonant acoustic mixing, were compared with BDDE-crosslinked hyaluronic acid obtained using standard chemical crosslinking techniques (Table 3). BDDE-crosslinked hyaluronic acid is the main ingredient in the most commonly used commercial dermal fillers (Table 2). The rheological properties of commercial dermal fillers such as Juvederm Ultra® XC, Juvederm Ultra Plus® XC, Juvederm Voluma® XC, Restylane Fynesse®, Restylane Kysse®, Restylane Volyme®, Restylane Refyne®, Restylane Silk®, Belotero Balance® were measured and compared to BDDE-crosslinked hyaluronic acid obtained using resonant acoustic mixing (Table 3).

TABLE 2
Commercial dermal fillers comprising
BDDE-crosslinked hyaluronic acid.

| Crosslinking Method | Product Code | Product Name |
|---|---|---|
| CPM ® (Cohesive | CPM-BB | Belotero Balance ® |
| Polydensified Matrix) | | |
| Hylacross ® | HYLJU | Juvederm Ultra ® XC |
| | HYL-JUP | Juvederm Ultra Plus ® XC |
| Vycross ® | VYC-VOLB | Juvederm Volbella ® |
| | VYC-VOLL | Juvederm Vollure ® |
| | VYC-VOLU | Juvederm Voluma ® XC |
| XpresHAn ® | XPRES-RF | Restylane Fynesse ® |
| | XPRES-RK | Restylane Kysse ® |
| | XPRES-RV | Restylane Volyme ® |
| | XPRES-RR | Restylane Refyne ® |
| | XPRES-RD | Restylane Defyne ® |
| NASHA ® (nonanimal | NASH-SLK | Restylane Silk ® |
| stabilized | NASH-R | Restylane ® |
| hyaluronic acid) | NASH-LYF | Restylane Lyft ® |
| RHA ® (Resilient | RHA-T1 | Teosyal RHA ® 1 |
| Hyaluronic Acid) | RHA-T2 | Teosyal RHA ® 2 |
| | RHA-T3 | Teosyal RHA ® 3 |
| | RHA-T4 | Teosyal RHA ® 4 |

TABLE 3
Rheological properties of dermal fillers comprising
BDDE-crosslinked hyaluronic acid and BDDE-crosslinked
hyaluronic acid obtained using resonant acoustic
mixing (i.e., ARC-02, ARC-06, ARC-07, ARC-08).

| # | Product | HA (mg/ml) | G′ (Pa) | G″ (Pa) | Tan δ | G* (Pa) |
|---|---|---|---|---|---|---|
| 1 | CPM-BB | 22.5 | 41 | 19 | 0.47 | 45 |
| 2 | HYLJU | 24 | 76 | 18 | 0.23 | 78 |
| 3 | HYL-JUP | 24 | 148 | 24 | 0.16 | 150 |
| 4 | VYC-VOLB | 15 | 159 | 21 | 0.13 | 161 |
| 5 | VYC-VOLL | 17.5 | 273 | 32 | 0.12 | 275 |
| 6 | VYC-VOLU | 20 | 307 | 29 | 0.09 | 308 |
| 7 | XPRES-RF | 20 | 10 | 5 | 0.52 | 11 |
| 8 | XPRES-RK | 20 | 156 | 12 | 0.07 | 156 |
| 9 | XPRES-RV | 20 | 150 | 11 | 0.08 | 150 |
| 10 | XPRES-RR | 20 | 47 | 7 | 0.16 | 48 |
| 11 | XPRES-RD | 20 | 260 | 16 | 0.06 | 260 |
| 12 | NASH-SLK | 20 | 344 | 79 | 0.23 | 353 |
| 13 | NASH-R | 20 | 544 | 99 | 0.18 | 553 |
| 14 | NASH-LYF | 20 | 545 | 69 | 0.13 | 549 |
| 15 | RHA-T1 | 15 | 48 | 21 | 0.44 | 52 |
| 16 | RHA-T2 | 23 | 144 | 36 | 0.25 | 148 |
| 17 | RHA-T3 | 23 | 184 | 29 | 0.16 | 186 |
| 18 | RHA-T4 | 23 | 296 | 37 | 0.12 | 298 |
| 19 | ARC-02 | 23 | 1810 | 163 | 0.09 | 1817 |
| 20 | ARC-06 | 18 | 392 | 46 | 0.12 | 395 |
| 21 | ARC-07 | 23 | 598 | 66 | 0.11 | 602 |
| 22 | ARC-08 | 23 | 519 | 45 | 0.09 | 521 |

The dynamic viscosity “n” of BDDE-crosslinked hyaluronic acids obtained using resonant acoustic mixing (ARC-02; ARC-06; ARC-07 and ARC-08) was subsequently compared with that of the commercial dermal fillers Teosyal RHA®1, RHA®2, RHA®3 and RHA®4 (RHA®—Resilient Hyaluronic Acid) (Table 4).

TABLE 4
Dynamic viscosity data.

| | Shear Rate, [1/s] | | |
|---|---|---|---|
| Gel ID | 1 | 10 | 50 |
| ARC-02 | 364.75 | 78.34 | 26.74 |
| ARC-06 | 148.94 | 51.98 | 11.53 |
| ARC-07 | 295.80 | 59.70 | 19.51 |
| ARC-08 | 269.15 | 56.62 | 19.05 |
| RHA-1 | | | 4.78 |
| RHA-2 | | | 7.66 |
| RHA-3 | | | 8.60 |
| RHA-4 | | | 9.10 |

The data illustrated herein demonstrate that crosslinked hyaluronic acids obtained by resonant acoustic mixing exhibit unique and advantageous rheological properties. To that effect, BDDE-crosslinked hyaluronic acids obtained by resonant acoustic mixing exhibit high dynamic viscosities (Table 4), making the material advantageously useful as a dermal filler. To that effect, the observed high dynamic viscosities provide for easy and smooth injection of the material, even when using high gauge needles. Moreover, BDDE-crosslinked hyaluronic acids obtained by resonant acoustic mixing exhibit advantageous elasticity relative to the best currently available commercial fillers. Furthermore, the use of resonant acoustic mixing to effect the crosslinking of hyaluronic acid provides for rapid reaction times, lower reaction temperatures, and substantially does way with isolation and purification procedures, characteristic of standard chemical crosslinking. However, a purification step and/or sterilization step may be incorporated into the process for preparing crosslinked polymers using resonant acoustic mixing. A further advantage of resonant acoustic mixing is that the rheological properties of the crosslinked material can be easily modified by changing the mixing parameters such as the forcing energy and/or frequency.

### General Methods and Materials

Reagents and solvents were obtained from commercial suppliers and used without further purification, unless otherwise noted. Resonant acoustic crosslinking was performed using a Resonant Acoustic Mixer LabRAM® | (Resodyn). Rheological properties were measure using an Anton-Paar Modular Compact Rheometer MCR® 502e (Ser. No. 81/046,409) using the RHEOPLUS®/32 Multi3 V3.62 software package.

All of the crosslinked polymers and/or processes disclosed and claimed herein can be made and executed without undue experimentation in light of the present disclosure. While the crosslinked polymers and/or processes of this disclosure have been described in terms of preferred embodiments, it will be apparent to those of skill in the art that variations may be applied to the crosslinked polymers and/or processes and in the steps or in the sequence of steps of the processes described herein without departing from the concept, spirit, and scope of the disclosure. More specifically, it will be apparent that certain agents which are chemically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

1. Samuel Gavard Molliard; Jérémie Bon Bétemps; Basste Hadjab; David Topchian; Patrick Micheels; Denis Salomon. Key rheological properties of hyaluronic acid fillers: from tissue integration to product degradation. Plast. Aesthet. Res. 2018; 5:17.
2. Jeffrey Kablik; Gary D. Monheit; Lipig Yu; Grace Chang; Julia Gershkovich. Comparative Physical Properties of Hyaluronic Acid Dermal Fillers. Dermatol. Surg. 2009; 35:302-312.
3. Stefano Santoro; Luisa Russo; Vincenzo Argenzio; Assunta Borzacchiello. Rheological properties of cross-linked hyaluronic acid dermal fillers. J. Appl. Biomater. Biomech. 2011; Vol. 9 no. 2, 127-136.
4. Steven Fagien; Vince Bertucci; Erika von Grote; Jay H. Mashburn. Rheologic and Physicochemical Properties Used to Differentiate Injectable Hyaluronic Acid Filler Products. Plast Reconstr. Surg. 2019, 143 (4): 707e-720e.
5. Yu Xue; Hongyue Chen; Chao Xu; Dinghua Yu; Huajin Xu; Yi Hu. Synthesis of hyaluronic acid hydrogels by crosslinking the mixture of high-molecular-weight hyaluronic acid and low-molecular-weight hyaluronic acid with 1,4-butanediol diglycidyl ether. RSC Adv., 2020, 10, 7206.

1. A process for crosslinking a polymer using resonant acoustic mixing, the process comprising:
adding an aqueous solution comprising a dissolved base to the polymer to produce a substantially homogenous gel;
adding a crosslinking agent to the substantially homogeneous gel to produce a mixture; and
subjecting the mixture to resonant acoustic mixing conditions sufficient to effect crosslinking of the polymer;
wherein the resonant acoustic mixing conditions comprise a forcing energy ranging between about 20 g to about 100 g.

2. The process of claim 1, wherein the resonant acoustic mixing conditions further comprise mixing frequencies ranging between about 40 Hz to about 90 Hz.

3. The process of claim 2, wherein the mixing frequency is about 60 Hz.

4. The process of any one of claims 1 to 3, wherein the resonant acoustic forcing energy ranges between about 60 g to about 80 g.

5. The process of any one of claims 1 to 4, wherein the substantially homogenous gel is produced following resonant acoustic mixing.

6. The process of claim 5, wherein the resonant acoustic mixing comprises a forcing energy ranging between about 20 g to about 100 g and mixing frequencies ranging between about 40 Hz to about 90 Hz.

7. The process of claim 6, wherein the mixing frequency is about 60 Hz.

8. The process of any one of claims 5 to 7, wherein the resonant acoustic forcing energy ranges between about 60 g to about 80 g.

9. The process of any one of claims 1 to 8, wherein the polymer is a carbohydrate polymer or a salt thereof.

10. The process of claim 9, wherein the carbohydrate polymer is hyaluronic acid or a salt thereof.

11. The process of any one of claims 1 to 10, wherein the crosslinking agent is a bifunctional crosslinking agent.

12. The process of claim 11, wherein the bifunctional crosslinking agent comprises 1,4-butanediol diglycidyl ether (BDDE).

13. The process of any one of claims 1 to 12, wherein the dissolved base comprises sodium hydroxide.

14. The process of any one of claims 1 to 13, wherein the addition steps are carried out at room temperature.

15. The process of claim 1, wherein the resonant acoustic mixing step is carried out at room temperature.

16. The process of any one of claims 1 to 15, wherein at least one of the process steps is carried out at room temperature.

17. The process of claim 1, wherein the resonant acoustic mixing is carried out over a period of time ranging from about 1 minute to about 10 minutes.

18. The process of any one of claims 1 to 17, further comprising a purification step and/or sterilization step.

19. The process of claim 18, wherein the purification step comprises washing the crosslinked polymer using an aqueous hydrochloric acid solution.

20. A product comprising a crosslinked polymer produced by resonant acoustic mixing.

21. The product of claim 20, wherein the crosslinked polymer is crosslinked hyaluronic acid.

22. The product of claim 20 or 21, wherein the product is a dermal filler.

23. The product of claim 22, wherein the dermal filler is an injectable dermal filler.

24. Use of the injectable dermal filler as defined in claim 23 for cosmetic applications.

25. The use of claim 24, wherein the cosmetic applications comprise treating and/or reducing the appearance of fine lines or wrinkles, glabellar lines, nasolabial folds, chin folds, marionette lines, jawlines, perioral wrinkles, crow's feet, oral commissures, cutaneous depressions, scars, temples, malar and buccal fat pads, tear troughs and facial asymmetries.

26. Use of the injectable dermal filler as defined in claim 23 for subdermal support of the brows, nose, lips, cheeks, chin, perioral region and/or infraorbital region.

27. Use of the product as defined in claim 20 for therapeutic and/or prophylactic applications.

28. The use of claim 27, wherein the therapeutic and/or prophylactic applications comprise stress urinary incontinence, vesicoureteral reflux (VUR), vocal fold insufficiency, and vocal fold medialization.

29. A method for treating a biological tissue or for increasing the volume of the biological tissue, the method comprising administering to a subject in need thereof an effective amount of the injectable dermal filler as defined in claim 23.

30. A process for crosslinking a polymer using resonant acoustic mixing, the process comprising subjection a mixture comprising the polymer and a crosslinking agent to resonant acoustic mixing conditions sufficient to effect crosslinking of the polymer.

31. The process of claim 30, further comprising adding an aqueous solution comprising a dissolved base to the polymer followed by resonant acoustic mixing to produce a substantially homogenous gel, and adding a crosslinking agent to the substantially homogeneous gel to produce the mixture.

32. The process of claim 31, wherein the dissolved base comprises sodium hydroxide.

33. The process of claim 30, wherein the resonant acoustic mixing conditions comprise a forcing energy ranging between about 20 g to about 100 g.

34. The process of claim 33, wherein the resonant acoustic forcing energy ranges between about 60 g to about 80 g.

35. The process of claim 33 or 34, wherein the resonant acoustic mixing conditions further comprise mixing frequencies ranging between about 40 Hz to about 90 Hz.

36. The process of claim 35, wherein the mixing frequency is about 60 Hz.

37. The process of any one of claims 30 to 36, wherein the polymer is a carbohydrate polymer or a salt thereof.

38. The process of claim 37, wherein the carbohydrate polymer is hyaluronic acid or a salt thereof.

39. The process of any one of claims 30 to 38, wherein the crosslinking agent is a bifunctional crosslinking agent.

40. The process of claim 39, wherein the bifunctional crosslinking agent comprises 1,4-butanediol diglycidyl ether (BDDE).

41. The process of claim 30, wherein the resonant acoustic mixing is carried out at room temperature.

42. The process of claim 30, wherein the resonant acoustic mixing is carried out over a period of time ranging from about 1 minute to about 10 minutes.

43. The process of any one of claims 30 to 42, further comprising a purification step and/or sterilization step.

44. The process of claim 43, wherein the purification step comprises washing the crosslinked polymer using an aqueous hydrochloric acid solution.

45. A gel comprising a crosslinked polymer produced by resonant acoustic mixing.

46. The gel of claim 45, wherein the crosslinked polymer is crosslinked hyaluronic acid.
